(19) United States
(12) Patent Application Publication
(10) Pub. No.: US 2022/0249204 A1
(43) Pub. Date: Aug. 11, 2022

(54) Mouth Guard System and Method of Use

(71) Applicant: Fahed Yusef Simon Tuma, Richmond, CA (US)

(72) Inventor: Fahed Yusef Simon Tuma, Richmond, CA (US)

(21) Appl. No.: 17/591,655

(22) Filed: Feb. 3, 2022

Related U.S. Application Data
(60) Provisional application No. 63/146,889, filed on Feb. 8, 2021.

(51) Int. Cl. A61C 7/08; A61C 7/00; A61C 9/00; G06T 7/00

(52) U.S. Cl. CPC A61C 7/08; A61C 7/002; G06T 2207/30036; G06T 7/0016; A61C 9/00

(57) ABSTRACT

The present invention relates generally to the field of mouthguards. More specifically, the present invention relates to a mouth guard system and method of use. The system is primarily comprised of a kit, at least one mouthguard, a mobile application and a method of use. The mouthguard of the system is preferably manufactured from a rigid, transparent, BPA-free material and can be worn during sleep to correct malocclusion to improve and prevent postural dysfunction, tensions headaches and other body ailments. The method of use further involves using a mold to create a custom mouthguard for a user, wherein the user can then wear the mouthguard and track postural changes induced by the mouthguard via a mobile application.

### CROSS-REFERENCE TO RELATED APPLICATION

The present application claims priority to, and the benefit of, U.S. Provisional Application No. 63/146,889, which was filed on Feb. 8, 2021, and is incorporated herein by reference in its entirety.

### FIELD OF THE INVENTION

The present invention relates generally to the field of mouthguards. More specifically, the present invention relates to a mouth guard system and method of use. The system is primarily comprised of a kit, at least one mouthguard, a mobile application and a method of use. The mouthguard of the system is preferably manufactured from a rigid, transparent BPA-free material and can be worn during sleep to correct malocclusion to improve and prevent postural dysfunction, tensions headaches and other body ailments. The method of use further involves using a mold to create a custom mouthguard for a user, wherein the user can then wear the mouthguard and track postural changes induced by the mouthguard via a mobile application. Accordingly, the present disclosure makes specific reference thereto. Nonetheless, it is to be appreciated that aspects of the present invention are also equally applicable to other like applications, devices and methods of manufacture.

### BACKGROUND

Malocclusion can lead to abnormal tension in certain muscles that can lead to painful tension headaches. In addition, since there is a relationship between the teeth, jaw joints, head, neck muscles, and head posture, one problem in any of said areas (such as muscle tension) may affect and cause problems in said other areas. These problems can include various types of head and neck pain, temporomandibular disorders, postural problems, excessive snoring, sleep apnea, etc.

More specifically, postural dysfunction (i.e., bad posture) occurs when the spine is positioned in unnatural positions. As a result, the joints, muscles, and vertebrae remain in stressful positions. Numerous individuals suffer painful negative effects of bad posture such as, but not limited to, body aches, upper or lower back pain, neck pain, shoulder pain, arm pain, tight neck muscles, the compromise of the natural curvature of the spine, etc., without making any change to the factors or habits that cause the postural dysfunction. Bad posture can further lead to the temporomandibular joint syndrome, wherein changes in body posture may affect the mandibular position. Said postural dysfunction can occur as a result of malocclusion and the tension caused by malocclusion on various muscles of the body.

Therefore, there exists a long-felt need in the art for a system that eliminates health problems such as head, shoulder, back, and body pain due to malocclusion. More specifically, there exists a long-felt need in the art for a mouth guard system and method of use that relieves tension in the head, back and body of a user by improving body posture of the user. There also exists a long-felt need in the art for a mouth guard system and method of use that aligns and relaxes the jaw of the user to aid in relaxing the muscles of the head, neck and body to relieve tension headaches, muscle aches, back problems, etc. Furthermore, there exists a long-felt need in the art for a mouth guard system and method of use that improves and/or prevents postural dysfunction.

The subject matter disclosed and claimed herein, in one embodiment thereof, comprises a mouth guard system and method of use. The system of the present invention is primarily comprised of a kit, at least one mouthguard, a mobile application and a method of use. The mouthguard of the system is preferably manufactured from a rigid, transparent, BPA-free material and can be worn during sleep to correct malocclusion, to improve and prevent postural dysfunction, tension headaches and other body ailments. The method of use further involves using a mold to create a custom mouthguard for a user, wherein the user can then wear the mouthguard and track postural changes induced by the mouthguard via a mobile application.

In this manner, the mouth guard system and method of use of the present invention accomplishes all of the forgoing objectives and relieves tension in the head, back and body of a user by improving the body posture of the user. The device also aligns and relaxes the jaw of the user to aid in relaxing the muscles of the head, neck and body to relieve tension headaches, muscle aches, back problems, etc. As a result, the device improves and/or prevents postural dysfunction.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a mouth guard system and method of use. The system of the present invention is primarily comprised of a kit, at least one mouthguard, a mobile application and a method of use. The mouthguard of the system is preferably manufactured from a rigid, transparent, BPA-free material. The mouthguard may be designed to be worn on the bottom teeth and/or top teeth of a user and is custom-fitted and shaped, such that any malocclusion of the user's bite is mitigated and/or corrected by the wearing of the mouthguard during sleep. In this manner, the mouthguard improves body posture by aligning and relaxing the jaw, relaxing the muscles of the head and body to relieve and preventing tension headaches, muscle aches, back problems and preventing or improving postural dysfunctions.

The system is further comprised of a method of use. A user first purchases a kit and creates an account with a username and password using the mobile application. The components of the kit include at least one bite mold, a set of instructions and a shipping package. Using the set of instructions, the user takes a mold of their mouth. Using the shipping package, the bite molds can then be shipped to a dental lab or other manufacturer who creates the mouthguard in the precise shape and dimensions of the user's bite, mouth and tooth size. Then, the mouthguard is sent to the user and can be worn overnight by the user while sleeping to eliminate malocclusion and to prevent or eliminate postural dysfunction.

In order to track any postural changes induced by the mouthguard, the system is also comprised of a mobile application that can be used on any smart device such as, but not limited to, a mobile phone, tablet, computer, etc. The application allows a user to track postural and bite improvements from the wearing the mouthguard. Using the application, the user first signs in to his or her account with his or her username and password and can then upload a plurality of photos that can be taken before, during (at differing stages), and after using the mouthguard. Therefore, the user can analyze the differences in posture via comparing the photos within the application.

Accordingly, the mouth guard system and method of use of the present invention is particularly advantageous as it relieves tension in the head, back and body of a user by improving the body posture of the user and aligns and relaxes the jaw of the user to aid in relaxing the muscles of the head, neck and body to relieve tension headaches, muscle aches, back problems, etc. Furthermore, the device improves and/or prevents postural dysfunction. In this manner, the mouth guard system and method of use provides a solution to said health problems.

To the accomplishment of the foregoing and related ends, certain illustrative aspects of the disclosed innovation are described herein in connection with the following description and the annexed drawings. These aspects are indicative, however of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a graphical view of one potential embodiment of a mouth guard system and method of use of the present invention in accordance with the disclosed architecture;

FIG. 2 illustrates a perspective view of a mouthguard of one potential embodiment of a mouth guard system and method of use of the present invention in accordance with the disclosed architecture;

FIG. 3 illustrates a graphical flow chart of a method of use of one potential embodiment of a mouth guard system and method of use of the present invention in accordance with the disclosed architecture;

FIG. 4 illustrates a graphical view of a kit of one potential embodiment of a mouth guard system and method of use of the present invention in accordance with the disclosed architecture; and

FIG. 5 illustrates a graphical view of a mobile application of one potential embodiment of a mouth guard system and method of use of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a system that eliminates health problems such as head, shoulder, back and body pain due to malocclusion. More specifically, there exists a long-felt need in the art for a mouth guard system and method of use that relieves tension in the head, back and body of a user by improving body posture of the user. There also exists a long-felt need in the art for a mouth guard system and method of use that aligns and relaxes the jaw of the user to aid in relaxing the muscles of the head, neck, and body to relieve tension headaches, muscle aches, back problems, etc. Furthermore, there exists a long-felt need in the art for a mouth guard system and method of use that improves and/or prevents postural dysfunction.

The present invention, in one exemplary embodiment, is comprised of a mouth guard system and method of use primarily comprised of a kit, at least one mouthguard, a mobile application and a method of use. The mouthguard of the system is preferably manufactured from a rigid, transparent, BPA-free material and may be designed to be worn on the bottom teeth and/or top teeth of a user and is custom-fitted and shaped such that any malocclusion of the user's bite is mitigated and/or corrected by the wearing of the mouthguard during sleep. In doing so, the mouthguard improves body posture by aligning and relaxing the jaw, relaxing the muscles of the head and body to relieve and prevent tension headaches, muscle aches, back problems and postural dysfunctions.

The system is further comprised of a method of use, wherein a user first purchases a kit and creates an account with a username and password using the mobile application. The components of the kit include at least one bite mold, a set of instructions, and a shipping package. Using the set of instructions, the user takes a mold of their mouth. Using the shipping package, the bite molds can then be shipped to a dental lab or other manufacturer who creates the mouthguard in the precise shape and dimensions of the user's bite, mouth and tooth size. Then, the mouthguard is sent to the user and can be worn overnight by the user while sleeping to eliminate malocclusion and to prevent or eliminate postural dysfunction.

The system is also comprised of a mobile application that can be used on any smart device such as, but not limited to, a mobile phone, tablet, computer, etc., wherein the application allows a user to track postural and bite improvements from the wearing the mouthguard. Using the application, the user first signs in to his or her account with his or her username and password and can then upload a plurality of photos that can be taking before, during (at differing stages) and after using the mouthguard. Therefore, the user can analyze the differences in posture via comparing the photo within the application.

Accordingly, the mouth guard system and method of use of the present invention is particularly advantageous as it relieves tension in the head, back and body of a user by improving the body posture of the user and aligns and relaxes the jaw of the user to aid in relaxing the muscles of the head, neck and body to relieve tension headaches, muscle aches, back problems, etc. Furthermore, the device improves and/or prevents postural dysfunction. In this manner, the mouth guard system and method of use provides a solution to said health problems.

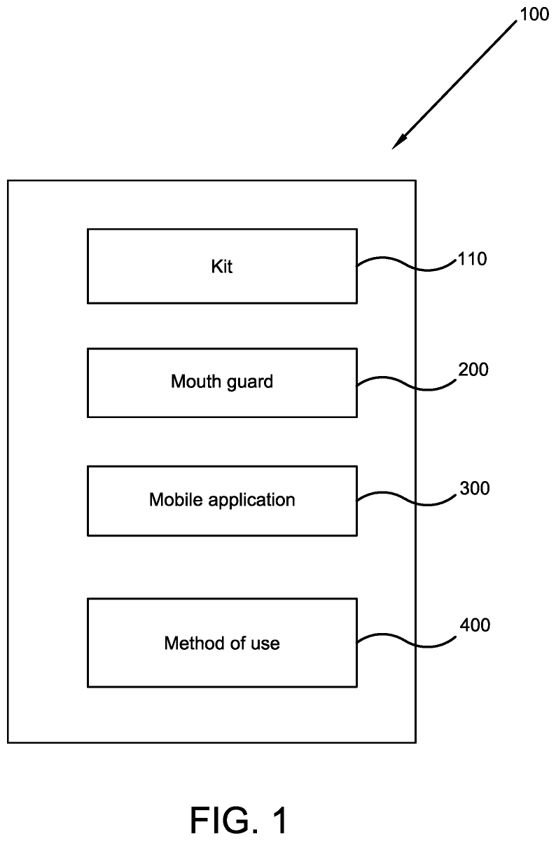

Referring initially to the drawings, FIG. 1 illustrates a graphical view of one potential embodiment of a mouth guard system and method of use 100 of the present invention in accordance with the disclosed architecture. The mouth guard system 100 of the present invention is primarily comprised of a kit 110, at least one mouthguard 200, a mobile application 300 and a method of use 400. The mouthguard 200 of the system 100 is preferably manufactured from a rigid, transparent BPA-free material. Said material can include, but is not limited to, an acrylic, polycarbonate, polyethylene, thermoplastic, acrylonitrile butadiene styrene, low density polyethylene, medium density polyethylene, high density polyethylene, polyethylene terephthalate, polyvinyl chloride, polystyrene, polylactic acid, acetal, nylon, fiberglass, recycled plastic, biodegradable plastic, etc. The mouthguard 200 may further be any color in the art and may be semi-transparent or opaque in other embodiments. In addition, the mouthguard 200 may be comprised of a plurality of indicia 230 such as, but not limited to, patterns, logos, emblems, images, symbols, designs, letters, numbers, words, characters, animals, advertisements, brands, etc. In other embodiments, the mouthguard 200 may also be flexible or semi-flexible.

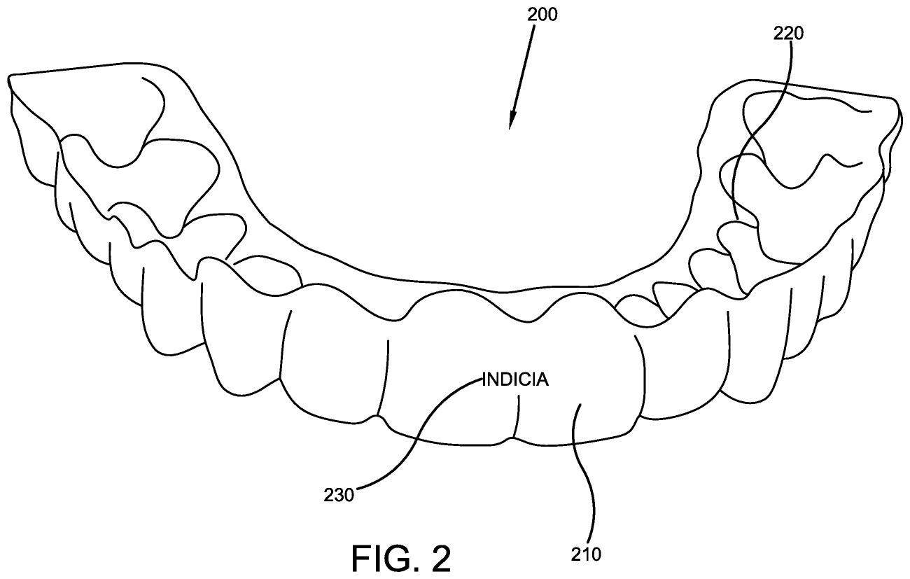

FIG. 2 illustrates a perspective view of a mouthguard 200 of one potential embodiment of a mouth guard system and method of use 100 of the present invention in accordance with the disclosed architecture. In differing embodiments, the mouthguard 200 may be designed to be worn on the bottom teeth and/or top teeth of a user. By way of example, if the mouthguard 200 is worn on the bottom teeth of a user, the inside surface 220 of the mouthguard 200 contacts the bottom teeth of the user and the outside surface 210 would contact the bottom of the top teeth of the user. Unlike regular mouthguards, which are used to protect teeth from bruxism while sleeping or from blunt force injuries, the mouthguard 200 of the present invention is custom-fitted and shaped such that any malocclusion of the user's bite is mitigated and/or corrected by the wearing of the mouthguard 200 during sleep. In this manner, the mouthguard 200 improves body posture by aligning and relaxing the jaw, relaxing the muscles of the head and body to relieve and prevent tension headaches, muscle aches, back problems and preventing or improving postural dysfunctions. However, the mouthguard 200 may also be used to protect teeth from bruxism, avoid lesions, protect teeth from blunt force injuries, and can be used by users who suffer from temporomandibular joint disorders. Although, it should be noted that the main purpose of the mouthguard 200 is to improve and correct postural dysfunction to avoid chronic health conditions.

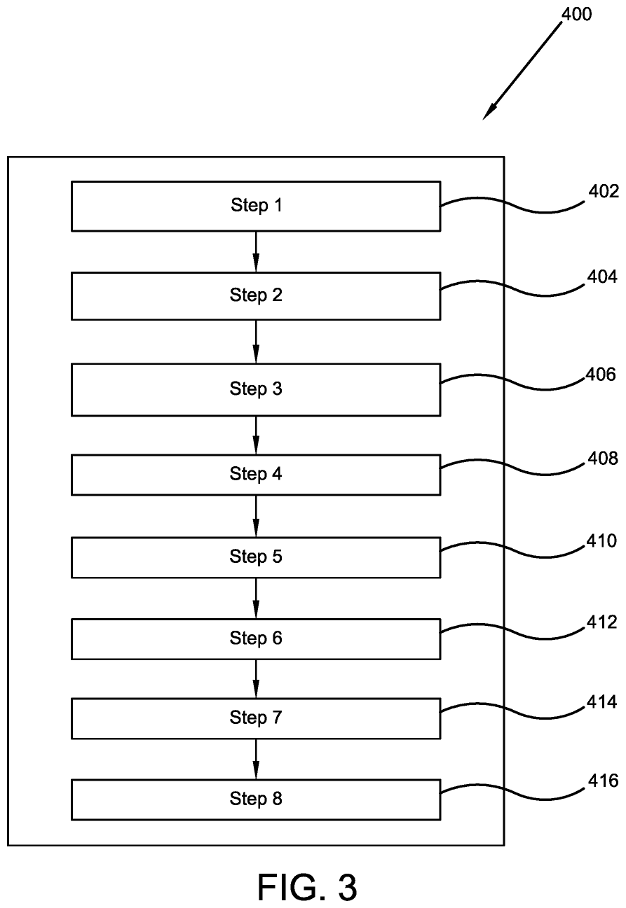

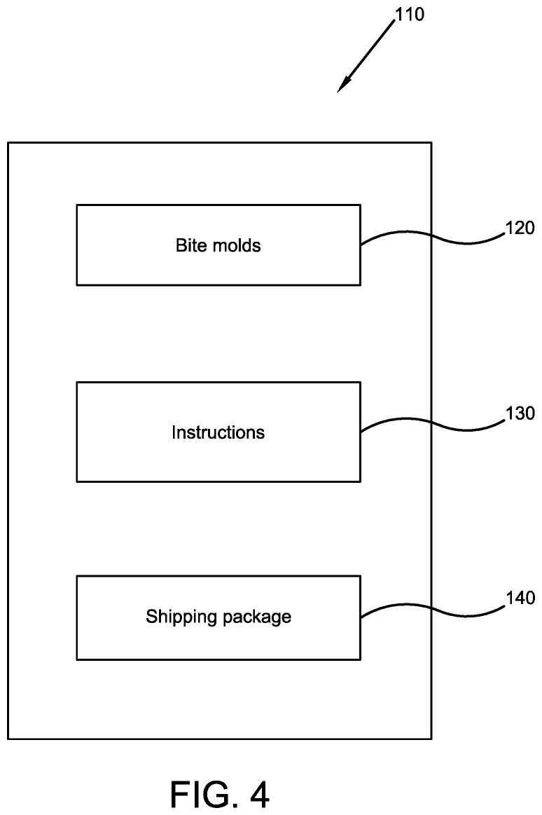

FIG. 3 illustrates a graphical flow chart of a method of use 400 of one potential embodiment of a mouth guard system and method of use 100 of the present invention in accordance with the disclosed architecture. The system 100 is further comprised of a method of use 400. It should be first noted that each mouthguard 200 is custom manufactured to conform to the unique shape of the user's mouth and location of the user's teeth in order to adequately correct any malocclusion of the user's bite. Therefore, a user first purchases a kit 110 and creates an account 310 with a username and password using the mobile application 300 [Block 402]. The components of the kit 110, as shown in FIG. 4, includes at least one bite mold 120, a set of instructions 130 and a paper or cardboard shipping package 140 such as, but not limited to, a box or envelope. The bite mold 120 is preferably manufactured from a condensation cure silicone impression putty or other similar dental impression putty of the like. Using the set of instructions 130, the user takes a mold of his or her mouth, wherein the mold 120 forms to all the crevices of each tooth and creates an exact mold of the user's bite [Block 404]. Using a shipping package 140 that is included in the kit 110, the bite molds 120 can then be shipped to a dental lab or other manufacturer [Block 406]. The lab or manufacturer then creates the mouthguard 200 via layering superheated plastic (or other material as noted above) to the precise shape and dimensions of the user's bite, mouth and tooth size. Then, the mouthguard 200 is sent to the user [Block 408]. Once received by the user, the mouthguard 200 can be worn overnight by the user while sleeping to eliminate malocclusion and to prevent or eliminate postural dysfunction [Block 410]. The mouthguard 200 will also enhance athletic performance.

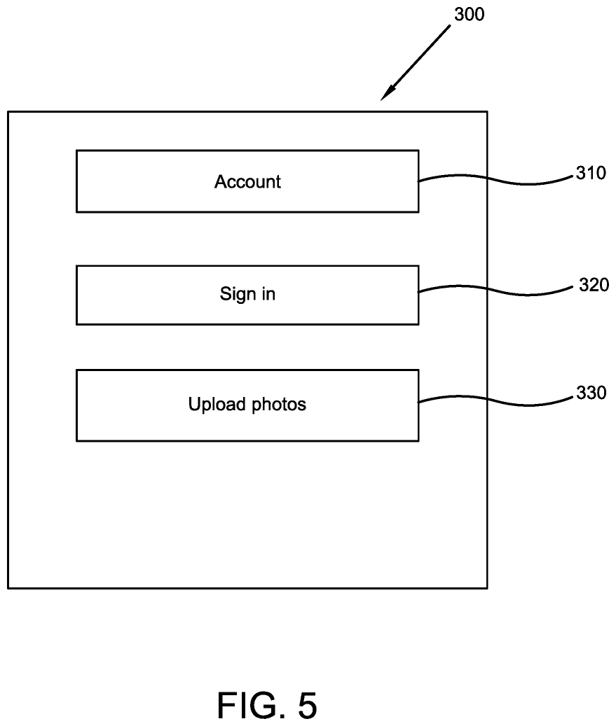

In order to track any postural changes induced by the mouthguard 200, the system 100 is also comprised of a mobile application 300 that can be used on any smart device such as, but not limited to, a mobile phone, tablet, computer, etc. The application 300, whose primary features are shown in FIG. 5, allows a user to track postural and bite improvements from the wearing the mouthguard 200. Using the application 300, the user first signs in using a sign in feature 320 to his or her account 310 with his or her username and password [Block 412]. Then, the user can upload a plurality of photos using a photo upload feature 330 that can be taken before, during (at differing stages), and after using the mouthguard 200 [Block 414]. In this manner, the user can analyze the differences in posture via comparing the photos 330 within the application 300 [Block 416].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “mouth guard system and method of use”, “mouth guard system”, and “system” are interchangeable and refer to the mouth guard system and method of use 100 of the present invention.

Notwithstanding the forgoing, the mouth guard system and method of use 100 of the present invention and its various components can be of any suitable size and configuration as is known in the art without affecting the overall concept of the invention, provided that they accomplish the above-stated objectives. One of ordinary skill in the art will appreciate that the size, configuration and material of the mouth guard system and method of use 100 as shown in the FIGS. are for illustrative purposes only, and that many other sizes and shapes of the mouth guard system and method of use 100 are well within the scope of the present disclosure. Although the dimensions of the mouth guard system and method of use 100 are important design parameters for user convenience, the mouth guard system and method of use 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A mouth guard system comprising:
a kit;
a mouthguard; and
a mobile application.

2. The mouth guard system of claim 1, wherein the mobile application is further comprised of an account, a sign in feature, and a photo upload feature.

3. The mouth guard system of use of claim 2, wherein the mobile application is installed on at least one of a mobile phone, a tablet and a computer.

4. The mouth guard system of use of claim 2, wherein a plurality of photos can be uploaded to the mobile application.

5. The mouth guard system of use of claim 1, wherein the kit is comprised of a bite mold, a set of instructions, and a shipping package.

6. The mouth guard system of use of claim 5, wherein the shipping package is a box or an envelope.

7. The mouth guard system of use of claim 5, wherein the bite mold is manufactured from a dental putty.

8. The mouth guard system of use of claim 1, wherein the mouthguard is comprised of an outside surface and an inside surface.

9. The mouth guard system of use of claim 8, wherein the mouthguard is manufactured from a rigid, transparent and BPA-free material.

10. A mouth guard system comprising:
a kit comprised of a bite mold, a set of instructions instructing a user how to utilize the bite mold and a shipping package;
a mouthguard comprised of an outside surface and an inside surface; and
a mobile application comprised of an account, a sign in feature, and a photo upload feature.

11. The mouth guard system of claim 10, wherein a plurality of photos can be uploaded to the mobile application.

12. The mouth guard system of claim 11, wherein the mobile application is installed on a select one of a mobile phone, a tablet or a computer.

13. The mouth guard system of claim 12, wherein the mouthguard is manufactured from a BPA-free material.

14. The mouth guard system of claim 13, wherein the bite mold is manufactured from a dental putty.

15. The mouth guard system of claim 14, wherein the shipping package is a box or an envelope.

16. A method of using a mouth guard system comprising:
purchasing a kit comprised of a set of instructions and a bite mold;
creating an account with a username and a password on a mobile application;
using the set of instructions and the bite mold of the kit to create a mouth mold;
using a shipping package to ship the mouth mold to a manufacturer who creates a mouthguard from the mouth mold;
shipping the mouthguard back to a user;
wearing the mouthguard while the user is sleeping; and
tracking a postural progress induced by the mouthguard by uploading a plurality of pictures via the mobile application.

17. The method of claim 16, wherein the shipping package is a box or an envelope.

18. The method of claim 16, wherein the mouthguard is manufactured from a rigid, transparent and BPA-free material.

19. The method of claim 16, wherein the bite mold is manufactured from a dental putty.

20. The method of use of claim 16, wherein the mobile application is installed on at least one of a mobile phone, a tablet and a computer.
